(19) United States
(12) Patent Application Publication
(10) Pub. No.: US 2023/0044952 A1
(43) Pub. Date: Feb. 9, 2023

(54) HONEY/SURFACTANT COMBINATIONS FOR WOUND TREATMENT

(71) Applicant: Derek T. Denton, Fair Oaks Rance, TX (US)

(72) Inventor: Derek T. Denton, Fair Oaks Rance, TX (US)

(21) Appl. No.: 17/878,324

(22) Filed: Aug. 1, 2022

Related U.S. Application Data
(60) Provisional application No. 63/228,867, filed on Aug. 3, 2021.

(51) Int. Cl. A61K 35/644; A61K 47/10; A61K 9/70; A61P 17/02

(52) U.S. Cl. CPC A61K 35/644; A61K 9/7023; A61K 47/10; A61P 17/02

(57) ABSTRACT

Natural honey and a surfactant are combined to achieve a novel wound treatment composition. The honey is chosen for its medicinal properties, and the surfactant is used for biofilm removal to enhance the effectiveness of the honey. In preferred embodiments, the honey is a tropical honey currently sourced from Mexico. The surfactant is mixed with water, then the aqueous mixture is combined with the honey. In accordance with a preferred embodiment, a surfactant powder is mixed with water at a ratio of 10% powder to water, then that mixture is combined with honey at a ratio greater than 50:50, more preferably at a ratio of 90:10. The currently preferred surfactant is Poloxamer 188. The product may be applied directly to a wound or applied to wound dressings including, without limitation, gauze, gauze rolls, hydrocolloids, and calcium alginate dressings.

### REFERENCE TO RELATED APPLICATIONS

This Application claims priority to, and the benefit of, U.S. Provisional Pat. Application Serial No. 63/228,867, filed Aug. 3, 2022, the entire content of which is incorporated herein by reference.

### FIELD OF THE INVENTION

This invention relates generally to wound treatment and, in particular, to a topical wound treatment dressing that combines tropical honey and a surfactant.

### BACKGROUND OF THE INVENTION

Many pharmaceuticals administered over the past several decades have had dangerous side effect and adverse drug interactions, including antibiotics that are now demonstrating more and more resistance to bacteria. For these reasons, many research groups are investigating the medicinal properties and mechanisms of many natural compounds, one of those being honey.

For over 5000 years, honey has been used to treat a variety of ailments, including acute and chronic wounds. Honey has anti-oxidant, anti-bacterial and anti-inflammatory properties. It can be used as a wound dressing to promote rapid and improved healing. These effects are due to honey’s anti-bacterial action (secondary to its high acidity), osmotic effect, anti-oxidant content and hydrogen peroxide content. The use of honey leads to improved wound healing in acute cases, pain relief in burn patients and decreased inflammatory response in such patients. Overall, numerous studies have been done in favor of the use of honey in wound care and medicine.

At the same time, the use of surfactants in wound care represents an emerging treatment for complicated or chronic wounds, due to their potential ability to prevent and breakdown recalcitrant biofilms. Biofilms are present in all chronic wounds, and are known to prevent wound closure by contributing to chronic inflammation, which increases a wound's propensity to infection. It has been shown that the presence of biofilms in wounds can reduce healing rates and increase the chances of infection leading to the formation of chronic wounds. Historically, biofilms were reported to be responsible for 65% of the bacterial infections present in chronic wounds.

Thus, biofilm removal may be essential to timely wound healing. Although the evidence demonstrating the effectiveness of surfactants is limited primarily to in vitro studies, there are a growing number of clinical studies demonstrating their ability to prevent biofilm formation and, consequently, enhance wound healing.

There are several examples of synthetic surfactants in wound care, with the most well-researched being poloxamers and betaines. Poloxamers are nonionic, synthetic surfactants composed on a central hydrophobic chain of polyoxypropylene and two hydrophilic chains of poloxyethylene. The chain length can be adjusted to produce different types of poloxamers. Poloxamer 188 is noted to have an inhibitory effect on biofilm formation in ex vivo porcine skin with either Staphylococcus aureus or Acinetobacter baumannii persisting in the wound following treatment Plurogel® (Medline Industries, Inc.) is an example of a wound gel containing the surfactant Poloxamer 188, which has shown its capability in reducing the inflammatory effects caused by biofilms by modulating the secretion of pro-inflammatory cytokines.

### SUMMARY OF THE INVENTION

This invention broadly combines natural honey and a surfactant to achieve a novel wound treatment composition. The honey is chosen for its medicinal properties, and the surfactant is used for biofilm removal to enhance the effectiveness of the honey. In preferred embodiments, the honey is a tropical honey currently sourced from Mexico.

The surfactant is mixed with water, then the aqueous mixture is combined with the honey. In accordance with a preferred embodiment, a surfactant powder is mixed with water at a ratio of 10% powder to water, then that mixture is combined with honey at a ratio greater than 50:50. In a more preferred embodiment, the ratio of honey to the aqueous surfactant mixture is 90:10; that is, 90% honey to a 10% surfactant/water mixture.

The currently preferred surfactant is Poloxamer 188, as it has been studied and proved effective. Other synthetic or natural surfactants are possible, and at different amounts when mixed with water. Similarly, other honey:surfactant mixture ratios are also possible but ratios with lesser amounts of honey we found to be “too liquid” and clinicians preferred the “handling” of a thicker consistency. The resultant combination may be provided as a solution in semi-liquid form, in jars and squeeze tubes, for example. The material may be applied directly to a wound or applied to wound dressings including, without limitation, gauze, gauze rolls, hydrocolloids, and calcium alginate dressings.

Utilizing a third-party FDA approved and A2LA accredited testing lab, the combination of a 90:10 mixture of Mexican tropical honey, and a solution comprising sterile water and ten percent (10%) Poloxamer 188 synthetic surfactant has shown tremendous and statistically significant 12 hr, 24 hr, 48 hr, and 72 hr kill results (ex vivo) on Acinetobacter baumannii, Staphylococcus aureus, E. Coli, MRSA, and Pseudomonas aeruginosa. Test results have been certified and documented by Adamson Analytical Laboratories of Corona, California.

### DETAILED DESCRIPTION OF THE INVENTION

Prior to this invention, honey and surfactants have been used separately for wound treatment. In particular, honey (with inert additives) and surfactants such as Poloxamer 188 (with inert additives) have been used separately to make different products. In accordance with this invention, however, tropical honey is combined with a surfactant for the first time to create a novel combination for wound care.

More specifically, this invention resides in a mixture of 100% medical grade honey and a solution comprised of sterile water and 10% Poloxamer 188, NF. The honey and P188 solution are measured out and combined at a contract manufacturing facility implementing FDA Good Manufacturing Procedures and Policies, and appropriate ISO 13485 certification to complete all manufacturing and packaging. Once packaged, the product will be shipped to a gamma radiation facility for sterilization and then will be ready for human use. The product can be offered in various sizes and containers; such as a 0.5, 1.5 and 6 oz. squeeze tubes.

### Example

1. Provide 100 percent (100%) tropical honey (currently sourced from Mexico);
2. Provide a solution of sterile water to ten percent (10%) Poloxamer 188 NF Powder;
3. Dilute the tropical honey and P188 solution in sterile water. The ratio of honey/P188 to water may be in any effective range, so far determined to be greater than 50%; more preferably 70%, and even more preferably 90%;
4. Optionally, an inert, water soluble, thickening agent may be added to the 10% Poloxamer P188 solution to thicken mixture to suit handling, application, and impregnation of dressings; and
5. The product is then packaged.

As mentioned, the anti-bacterial effect of honey kills bacteria present in acute and chronic wounds, while the surfactant inhibits biofilm growth and colonization. The beneficial role of honey is attributed to its antibacterial property in conjunction with its high osmolarity, acidity (low pH), content of hydrogen peroxide (H₂O₂) and non-peroxide components, i.e., the presence of phytochemical components.

The antimicrobial agents in honey are predominantly hydrogen peroxide, of which the concentration is determined by relative levels of glucose oxidase, synthesized by the bee and catalase originating from flower pollen. Most types of honey generate H₂O₂ when diluted, because of the activation of the enzyme glucose oxidase that oxidizes glucose to gluconic acid and H₂O₂, which promotes antimicrobial activity.

Poloxamer 188, which has shown its capability in reducing the inflammatory effects caused by biofilms by modulating the secretion of pro-inflammatory cytokines. Additionally, Poloxamer 407 has been reported to reduce biofilm formation by disrupting the attachment of Staphylococcus epidermidis to the wound surface. Consequently, poloxamers, in particular, represent potential effective surfactants capable of managing wound biofilms in conjunction with natural honey to promote faster healing.

Surfactants are able to disrupt biofilms and treat infection by various mechanisms. They can be left in situ for approximately 15 minutes to help loosen necrotic tissue or be scrubbed lightly on the wound surface with a sterile gauze. The micelles formed by the surfactant are able to change from a collapsed to an expanded state allowing wound debris to become trapped. This creates a rinsing action allowing the wound to be cleaned and preventing microbes from adhering to the wound surface, thus preventing biofilm formation. This rinsing action is also effective in removing older biofilms from the wound surface by disrupting the EPS, allowing the microbes to become more susceptible to the host's immune response and antibiotics.

In addition to the disruptive effects of surfactants on the biofilm’s structure, they can also be used as carriers for antimicrobials. Gel-based surfactants allow the antimicrobials to stay localized to the wound as they are more adhesive than liquids, hence, they allow the sustained delivery of antimicrobials to target the biofilm.

When a clinician is presented with an acute (sudden) or chronic wound (i.e., a wound that has not healed with normal interventions for 4-12 weeks), the wound should first be sharp or mechanically debrided and cleansed. Then a tropical honey/surfactant solutions according to the invention are administered with an applicator from a jar, or squeezed from a tube, and/or covered with a product-impregnated dressing or another appropriate applicator. Dressing changes may occur every 24, 48, or 72 hours as prescribed by the clinician. The process is repeated until wound closure. Products described herein may be applied to acute, chronic, and burn wounds.

### REFERENCES

Evidence for Clinical Use of Honey in Wound Healing as an Anti-bacterial, Anti-inflammatory Anti-oxidant and Anti-viral Agent: A Review. Jundishapur J Nat Pharm Prod. 2013 Aug; 8(3): 100-104. Published online 2013 Jul 17. doi: 10.17795/jjnpp-9487

Wounds International 2019 | Vol 10 Issue 1 | Wounds International 2019 Authors: Heather Charlotte Tyldesley, Anne-Marie Salisbury, Rui Chen, Marc Mullin and Steven L. Perci

Manisha Deb Mandal and Shyamapada Mandal /Asian Pac J Trop Biomed 2011; 1(2): 154-160

1. A method of making a wound dressing material, comprising:
proving honey;
providing a surfactant;
combining the honey and the surfactant; and
applying the combination to a wound.

2. The method of claim 1, wherein the surfactant is Poloxamer 188 (P188).

3. The method of claim 1, including the step of diluting the surfactant prior to combining the honey.

4. The method of claim 1, including the step of diluting the surfactant in water prior to combining the honey and the sulfactact.

5. The method of claim 1, including the step of diluting the surfactant in water at a ratio of 1 part surfactant to 9 parts water prior to the step of combining the honey and the surfactant.

6. The method of claim 3, wherein the diluted surfactant and honey are combined at a ratio of 9:1.

7. The method of claim 1, wherein the combination is applied directly to a wound.

8. The method of claim 1, wherein the combination is applied to a bandage, compress or other dressing adapted for wound contact.

9. The method of claim 1, wherein the honey is a tropical honey.

10. A wound treatment composition made in accordance with the method of claim 1.

11. A wound treatment dressing made in accordance with the method of claim 8.
